(19) United States
(12) Patent Application Publication
(10) Pub. No.: US 2024/0199730 A1
(43) Pub. Date: Jun. 20, 2024

(54) AN ANTI-TSLP FAB WITH IMPROVED STABILITY

(71) Applicant: MEDIMMUNE LIMITED, Cambridge, Cambridgeshire (GB)

(72) Inventors: Roland Wilhelm Kolbeck, Gaithersburg, MD (US); Emma Suzanne Cohen, Cambridge (GB); Catherine Eugenie Huntington, Cambridge (GB)

(21) Appl. No.: 18/287,240

(22) PCT Filed: Apr. 19, 2022
(86) PCT No.: PCT/EP22/60236

§ 371 Date: Oct. 17, 2023

(30) Foreign Application Priority Data
Apr. 19, 2021 (EP) 21169183.7

(51) Int. Cl. C07K 16/24

(52) U.S. Cl. CPC C07K 16/244; C07K 2317/21; C07K 2317/55

(57) ABSTRACT

The present disclosure relates to an anti-TSLP Fab with improved stability, nucleic acids encoding said Fab, host cells and vector comprising said nucleic acids, and methods of using said Fab in the treatment of TSLP-related conditions.

### TECHNICAL FIELD

The present disclosure relates to an anti-TSLP Fab with improved stability, nucleic acids encoding said Fab, host cells and vector comprising said nucleic acids, and methods of using said Fabs in the treatment of diseases.

### BACKGROUND

Thymic stromal lymphopoietin (TSLP) is a cytokine that signals through a heterodimeric receptor consisting of the IL-7Ra subunit and TSLP-R a unique component with homology to the common y-receptor-like chain (Pandey et al., Nat. Immunol. 2000, 1(1):59-64). TSLP is expressed by epithelial cells in the thymus, lung, skin, intestine, and tonsils, as well as airway smooth muscle cells, lung fibroblasts, and stromal cells (Edwards, 2008, Drug news & perspectives 21, 312-316; He and Geha, 2010, Annals of the New York Academy of Sciences 1183, 13-24; Reche et al., 2001, Journal of immunology 167, 336-343).

These cells produce TSLP in response to proinflammatory stimuli, and TSLP drives allergic inflammatory responses through its activity on a number of innate immune cells, including dendritic cells (Soumelis et al., 2002, Nature immunology 3, 673-680), monocytes (Reche et al., 2001, Journal of immunology 167, 336-343), and mast cells (Allakhverdi et al., 2007, The Journal of Experimental Medicine 204, 253-258). The cell populations with the highest known expression of both TSLP-R and IL-7Ra are myeloid dendritic cells (Reche et al., 2001, Journal of immunology 167, 336-343).

TSLP can promote proliferation of naive T cells and drive their differentiation into Th2 cells expressing high levels of iL-4, IL-5, and IL-13 (Omori and Ziegler, 2007, Journal of immunology 178, 1396-1404). High level of TSLP expression has been found in asthmatic lung epithelial cells and chronic atopic dermatitis lesions, suggesting a role for TSLP in allergic inflammation (Ziegler and Artis, 2010, Nature immunology 11, 289-293). More recent evidence implicates TSLP in the differentiation of Th17 cells and Th17-driven inflammatory processes (Hartgring et al., 2011, Arthritis and rheumatism 63, 1878-1887; Tanaka et al., 2009, Clinical and experimental allergy: Journal of the British Society for Allergy and Clinical Immunology 39, 89-100; Wu et al., 2014, Journal of molecular and cellular cardiology 76, 33-45). Chronic allergic (atopic) asthma is often characterized by Th2-type inflammation, while non-allergic asthmatic inflammation is predominately neutrophilic with a mixed Th1 and Th17 cytokine milieu. The consequences of chronic inflammation in asthma include bronchial hyper-reactivity (BHR), mucus overproduction, airway wall remodeling and airway narrowing (Lambrecht and Hammad, 2014, Nature immunology 16, 45-56). TSLP was shown to be involved in the initiation and maintenance/enhancement of the allergic asthmatic response (Wang et al., 2006, Immunity 24, 827-838). More recently, TSLP signaling was also found to be required for the recall response of memory T-cells to local antigen challenge (Wang et al., 2015, The Journal of allergy and clinical immunology 135, 781-791 e783).

Tezepelumab is an human immunoglobulin G2 (lgG2) monoclonal antibody (mAb) that binds to TSLP, preventing its interaction with the TSLP receptor complex. A proof-of concept study in patients with mild, atopic asthma, demonstrated that tezepelumab inhibited the early and late asthmatic responses and suppressed biomarkers of Th2 inflammation following inhaled allergen challenge. A study to evaluate tezepelumab in adults & adolescents with severe uncontrolled asthma (NCT03347279) recently concluded and met its primary endpoint of reducing annualized asthma exacerbation rate (AERR) [Time Frame: Baseline to Week 52].

CSJ117 is a potent neutralizing antibody fragment against human Thymic stromal lymphopoietin (TSLP), formulated as a PulmoSol™ engineered powder in hard capsules for delivery to the lungs via dry powder inhaler (Gauvrea et al ERS 2020 56: Suppl. 64, 3690).

Targeting TSLP in the treatment of inflammatory disorders such as asthma is therefore clinically validated. The administration of TSLP antagonists via inhalation is intriguing given that the majority of asthma sufferers are used to self-medicating via inhalation.

The present disclosure aims to build upon existing treatment options, particularly via inhalation in this emerging class of medicines.

### SUMMARY OF THE DISCLOSURE

The present disclosure has surprisingly found that making multiple mutations in the CH1 domain of Tezepelumab and converting the molecule to a Fab reduces aggregation under accelerated stability conditions (in 1×PBS, at 45ºC for two weeks). Accelerated stability studies in 1×PBS are sometimes used as a rough approximation of in vivo serum stability.

The majority of antibodies administered for treatments have half-lives in excess of 14 days (two weeks). Fabs may be preferred over mAbs for inhaled delivery given their smaller size and improved biodistribution in the lung following aerosolization. Despite being administered via inhalation, serum stability is still a relevant factor for protein-based antagonists administered to the lung, as the majority of active agent may ultimately partition into the serum. Therefore, the improved accelerated stability of the exemplary Fabs may be advantageous when used in the treatment of TSLP-related conditions.

Accordingly, in one aspect, the disclosure provides a Fab comprising a heavy chain comprising the amino acid sequence set forth in SEQ ID NO:1 and a light chain comprising the amino acid sequence set forth in SEQ ID NO:2.

In another aspect, the disclosure provides a Fab comprising a heavy chain having the amino acid sequence set forth in SEQ ID NO:1 and a light chain having the amino acid sequence set forth in SEQ ID NO:2.

In another aspect, the disclosure provides a Fab comprising a heavy chain consisting of the amino acid sequence set forth in SEQ ID NO:1 and a light chain consisting of the amino acid sequence set forth in SEQ ID NO:2.

In another aspect, the disclosure provides a nucleic acid encoding a Fab described herein.

In another aspect, the disclosure provides a vector comprising a nucleic acid described herein.

In another aspect, the disclosure provides a host cell comprising a nucleic acid or vector described herein.

In another aspect, the disclosure provides a method of treatment comprising administering to the subject a therapeutically effective amount of a Fab described herein.

In another aspect, the disclosure provides a Fab described herein for use in therapy.

In another aspect, the disclosure provides for the use of a Fab described herein in therapy.

In another aspect, the disclosure provides for the use of a Fab described herein in the manufacture of a medicament for use in therapy.

### BRIEF DESCRIPTION OF DRAWINGS

FIG. 1 shows the 2-week accelerated stability assay of ˜ 1 mg/ml Tezepelumab in 1×PBS at 4° C. (A) and 45° ° C. (B). The plot show HP-SEC traces of Tezepelumab following incubation at each temperature for the allotted time. The table (C) specifies the % monomer loss and % aggregate formation for Tezepelumab, together with results of IgG1 isotype control NIP228.

FIG. 2 shows the 2-week accelerated stability assay of ˜ 0.8 mg/ml Fab1 in 1×PBS at 4° C. (A) and 45° C. (B). The plot show HP-SEC traces of Fab1 following incubation at each temperature for the allotted time. The table (C) specifies the % monomer loss and % aggregate formation for Fab1, together with results of Fab isotype control R347.

FIG. 3 shows Fab 1 binding to hu TSLP as measured by KinExA.

FIG. 4 shows Fab₁ binding to cyno TSLP as measured by KinExA

FIG. 5 shows the competitive binding of Fab 1 to hu TSLP as measure using the HTRF assay

FIG. 6 shows that Fab 1 inhibits CCL17 release from PBMCs challenged with TSLP

### DETAILED DESCRIPTION

### Definition

It is to be noted that the term “a” or “an” entity refers to one or more of that entity; for example, “an anti-TSLP Fab” is understood to represent one or more anti-TSLP Fabs.

“Antibody” is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

“Antibody fragments” include antigen-binding portions of the antibody including, inter alia, Fab, Fab′, F(ab′)2, Fv, domain antibody (dAb), complementarity determining region (CDR) fragments, CDR-grafted antibodies, single-chain antibodies (scFv), single chain antibody fragments, chimeric antibodies, diabodies, triabodies, tetrabodies, minibody, linear antibody; chelating recombinant antibody, a tribady or bibody, an intrabody, a nanobody, a small modular immunopharmaceutical (SMIP), an antigen-binding-domain immunoglobulin fusion protein, single domain antibodies (including camelized antibody), a VHH containing antibody, or a variant or a derivative thereof, and polypeptides that contain at least a portion of an immunoglobulin that is sufficient to confer specific antigen binding to the polypeptide, such as one, two, three, four, five or six CDR sequences, as long as the antibody retains the desired biological activity.

“Fab” refers to an antibody fragment comprising the VH-CH1 and VL-CL pairing. The term encompasses Fabs comprising non-canonical sequence variants such as amino acid substitutions, deletions or insertions within the Fab outside of sequence regions typically associated with high sequence variability. For example, Fab variants include Fabs comprising non-canonical amino acid or sequence changes in VH or VL framework regions or in the CH1 or CL domains. Such changes may include the presence of non-canonical cysteines or other derivatizable amino acids, which may be used to conjugate said Fab variants to heterologous moieties. Other such changes include the presence of non-canonical polypeptide linkers, which are polypeptide sequences that covalently bridge between two domains. For example, a Fab variant may comprise a linker polypeptide that covalently attaches the CH1 domain to the VL domain, or the CL domain to the VH domain, such that the Fab can be expressed as a single polypeptide chain.

“Host cells” refers to cells that harbor vectors constructed using recombinant DNA techniques and encoding at least one heterologous gene. In descriptions of processes for isolation of anti-TSLP Fabs from recombinant hosts, the terms “cell” and “cell culture” are used interchangeably to denote the source of the anti-TSLP Fab unless it is clearly specified otherwise. In other words, recovery of polypeptide from the “cells” may mean either from spun down whole cells, or from the cell culture containing both the medium and the suspended cells.

“Isolated” refers to a polypeptide, antibody, polynucleotide, vector, cell, or composition which is in a form not found in nature. Isolated polypeptides, antibodies, polynucleotides, vectors, cells or compositions include those which have been purified to a degree that they are no longer in a form in which they are found in nature. In some aspects, an antibody, polynucleotide, vector, cell, or composition which is isolated is substantially pure.

“Pharmaceutical composition” refers to a preparation which is in such form as to permit the biological activity of the active ingredient (e.g., an anti-TSLP Fab disclosed herein) to be effective, and which contains no additional components which are unacceptably toxic to a subject to which the composition would be administered. Such composition can be sterile.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide can comprise modified nucleotides, such as methylated nucleotides and their analogs. The preceding description applies to all polynucleotides referred to herein, including RNA and DNA.

“Recombinant” polypeptide or protein refers to a polypeptide or protein produced via recombinant DNA technology. Recombinantly produced polypeptides and proteins expressed in engineered host cells are considered isolated for the purpose of the invention, as are native or recombinant polypeptides which have been separated, fractionated, or partially or substantially purified by any suitable technique. The polypeptides disclosed herein can be recombinantly produced using methods known in the art. Alternatively, the proteins and peptides disclosed herein can be chemically synthesized.

“Subject” or “individual” or “animal” or “patient” or “mammal,” is means any subject, particularly a mammalian subject, for whom diagnosis, prognosis, or therapy is desired, except where the subject is defined as a ‘healthy subject’. Mammalian subjects include humans; domestic animals; farm animals; such as dogs, cats, guinea pigs, rabbits, rats, mice, horses, cattle, cows, and so on. Preferably the subject is human.

“Treat” or “treatment” refer to both therapeutic treatment and prophylactic or preventative measures, wherein the object is to prevent or slow down (lessen) an undesired physiological change or disorder. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

“Therapeutically effective amount” refers to an amount of an anti-TSLP Fab disclosed herein or other drug effective to “treat” a disease or disorder in a subject or mammal.

“TSLP” refers to thymic stromal lymphopoietin. TSLP is a cytokine that signals through a heterodimeric receptor consisting of the IL-7Ra subunit and TSLP-R, a unique component with homology to the common g-receptor-like chain (Pandey et al., Nat. Immunol. 2000, 1(1):59-64). TSLP is expressed by epithelial cells in the thymus, lung, skin, intestine, and tonsils, as well as airway smooth muscle cells, lung fibroblasts, and stromal cells (Edwards, 2008, Drug news & perspectives 21, 312-316; He and Geha, 2010, Annals of the New York Academy of Sciences 1183, 13-24; Reche et al., 2001, Journal of immunology 167, 336-343). These cells produce TSLP in response to proinflammatory stimuli, and TSLP drives allergic inflammatory responses through its activity on a number of innate immune cells, including dendritic cells (Soumelis et al, 2002, Nature immunology 3, 673-680), monocytes (Reche et al., 2001, Journal of immunology 167, 336-343), and mast cells (Allakhverdi et al., 2007, The Journal of Experimental Medicine 204, 253-258). The cell populations with the highest known expression of both TSLP-R and IL-7Ra are myeloid dendritic cells (Reche et al, 2001, Journal of immunology 167, 336-343).

TSLP can promote proliferation of naive T cells and drive their differentiation into Th2 cells expressing high levels of IL-4, IL-5, and IL-13 (Omori and Ziegler, 2007, Journal of immunology 178, 1396-1404). High level of TSLP expression has been found in asthmatic lung epithelial cells and chronic atopic dermatitis lesions, suggesting a role for TSLP in allergic inflammation (Ziegler and Artis, 2010, Nature immunology 11, 289-293). More recent evidence implicates TSLP in the differentiation of Th17 cells and Th17-driven inflammatory processes (Hartgring et al, 2011, Arthritis and rheumatism 63, 1878-1887; Tanaka et al, 2009, Clinical and experimental allergy: Journal of the British Society for Allergy and Clinical Immunology 39, 89-100; Wu et al, 2014, Journal of molecular and cellular cardiology 76, 33-45). Chronic allergic (atopic) asthma is often characterized by Th2-type inflammation, while non-allergic asthmatic inflammation is predominately neutrophilic with a mixed Th1 and Th17 cytokine milieu. The consequences of chronic inflammation in asthma include bronchial hyper-reactivity (BHR), mucus overproduction, airway wall remodeling and airway narrowing (Lambrecht and Hammad, 2014, Nature immunology 16, 45-56). TSLP was shown to be involved in the initiation and maintenance/enhancement of the allergic asthmatic response (Wang et al., 2006, Immunity 24, 827-838). More recently, TSLP signaling was also found to be required for the recall response of memory T-cells to local antigen challenge (Wang et al., 2015, The Journal of allergy and clinical immunology 135, 781-791 e783).

“Vector” means a construct, which is capable of delivering, and in some aspects, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

### Anti-TSLP Fabs

The disclosure provides a Fab comprising a heavy chain comprising the amino acid sequence set forth in SEQ ID NO:1 and a light chain comprising the amino acid sequence set forth in SEQ ID NO:2.

In another aspect, the disclosure provides a Fab comprising a heavy chain comprising the amino acid sequence set forth in SEQ ID NO:1 and a light chain comprising the amino acid sequence set forth in SEQ ID NO:2.

In another aspect, the disclosure provides a Fab comprising a heavy chain comprising the amino acid sequence set forth in SEQ ID NO:1 and a light chain comprising the amino acid sequence set forth in SEQ ID NO:2.

The examples show that a Fab with these sequence characteristics has improved stability under accelerated stability study conditions compared to a full-length antibody with similar VH and VL domain sequences. The exemplary Fab with improved stability is referred to herein as Fab 1.

In some instances, the disclosure provides a Fab which has equivalent stability to Fab 1. In some instances, the equivalent stability is equivalent stability under accelerated stability study conditions. In some instances, accelerated stability study conditions are 1×PBS at 45° C. for two weeks. In some instances, the concentration of Fab under accelerated study conditions is ˜0.8 mg/ml.

In some instances, the Fab comprises a heavy chain having at least 80%, 85%, 90% or 95% sequence identity to the amino acid sequence of SEQ ID NO:1. In some instances, the Fab comprises a heavy chain having at least 95% sequence identity to the amino acid sequence of SEQ ID NO:1. In some instances, the Fab comprises equivalent stability to Fab 1.

In some instances, the Fab comprises a light chain having at least 80%, 85%, 90% or 95% sequence identity to the amino acid sequence of SEQ ID NO:2. In some instances, the Fab comprises a light chain having at least 95% sequence identity to the amino acid sequence of SEQ ID NO:2. In some instances, the Fab comprises equivalent stability to Fab 1.

In some instances, the Fab comprises a heavy chain having at least 80%, 85%, 90% or 95% sequence identity to the amino acid sequence of SEQ ID NO:1 and a light chain having at least 80%, 85%, 90% or 95% sequence identity to the amino acid sequence of SEQ ID NO:2. In some instances, the Fab comprises equivalent stability to Fab 1.

In some instances, the Fab comprises a heavy chain having at least 95% sequence identity to the amino acid sequence of SEQ ID NO:1 and a light chain having at least 95% sequence identity to the amino acid sequence of SEQ ID NO:2. In some instances, the Fab comprises equivalent stability to Fab 1.

In some instances, the disclosure provides an antigen binding fragment comprising a heavy chain of SEQ ID NO:1.

In some instances, the disclosure provides the antigen binding fragment comprises a heavy chain having at least 80%, 85%, 90% or 95% sequence identity to the amino acid sequence of SEQ ID NO:1. In some instances, the antigen binding fragment comprises a heavy chain having at least 95% sequence identity to the amino acid sequence of SEQ ID NO:1. In some instances, the antigen binding fragment comprises equivalent stability to Fab 1.

In some instances, the antigen binding fragment comprises a light chain having at least 80%, 85%, 90% or 95% sequence identity to the amino acid sequence of SEQ ID NO:2. In some instances, the antigen binding fragment comprises a light chain having at least 95% sequence identity to the amino acid sequence of SEQ ID NO:2. In some instances, the antigen binding fragment comprises equivalent stability to Fab 1.

In some instances, the antigen binding fragment comprises a heavy chain having at least 80%, 85%, 90% or 95% sequence identity to the amino acid sequence of SEQ ID NO:1 and a light chain having at least 80%, 85%, 90% or 95% sequence identity to the amino acid sequence of SEQ ID NO:2. In some instances, the antigen binding fragment comprises equivalent stability to Fab 1.

In some instances, the antigen binding fragment comprises a heavy chain having at least 95% sequence identity to the amino acid sequence of SEQ ID NO:1 and a light chain having at least 95% sequence identity to the amino acid sequence of SEQ ID NO:2. In some instances, the antigen binding fragment comprises equivalent stability to Fab 1.

### Nucleotides

The present disclosure also provides for nucleic acids (or “polynucleotides”) that encode Fabs and antigen-binding fragments disclosed herein.

The polynucleotides disclosed herein may further include additional nucleic acids, encoding, e.g., a signal peptide to direct secretion of the encoded polypeptide described herein.

The polynucleotides may be produced or manufactured by any method known in the art. For example, if the nucleotide sequence of the Fab is known, a polynucleotide encoding the anti-TSLP Fab may be assembled from chemically synthesized oligonucleotides (e.g., as described in Kutmeier et al., Bio Techniques 17:242 (1994)), which, briefly, involves the synthesis of overlapping oligonucleotides containing portions of the sequence encoding the anti-TSLP Fab, annealing and ligating of those oligonucleotides, and then amplification of the ligated oligonucleotides by PCR.

Alternatively, a polynucleotide encoding an anti-TSLP Fab, may be generated from nucleic acid from a suitable source. If a clone containing a nucleic acid encoding a Fab is not available, but the sequence of the Fab is known, a nucleic acid encoding the Fab may be chemically synthesized or obtained from a suitable source e.g., by PCR amplification using synthetic primers hybridizable to the 3′ and 5′ ends of the sequence or by cloning using an oligonucleotide probe specific for the particular sequence of interest. Amplified nucleic acids generated by PCR may then be cloned into replicable cloning vectors using any method well known in the art.

Once the nucleotide sequence and corresponding amino acid sequence of the Fab is determined, its nucleotide sequence may be manipulated using methods well known in the art for the manipulation of nucleotide sequences, e.g., recombinant DNA techniques, site directed mutagenesis, PCR, etc. (see, for example, the techniques described in Sambrook et al. (1990) Molecular Cloning, A Laboratory Manual (2nd ed.; Cold Spring Harbor Laboratory, Cold Spring Harbor, N.Y.) and Ausubel et al., eds. (1998) Current Protocols in Molecular Biology (John Wiley & Sons, NY), which are both incorporated by reference herein in their entireties), to generate Fabs having a different amino acid sequence, for example to create amino acid substitutions, deletions, and/or insertions.

A polynucleotide encoding a Fab can be composed of any polyribonucleotide or polydeoxyribonucleotide, which may be unmodified RNA or DNA or modified RNA or DNA. For example, a polynucleotide encoding an anti-TSLP Fab can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, a polynucleotide encoding said Fab can be composed of triple-stranded regions comprising RNA or DNA or both RNA and DNA. A polynucleotide encoding an anti-TSLP Fab may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

An isolated polynucleotide encoding a non-natural variant of a polypeptide derived from an immunoglobulin (e.g., an immunoglobulin heavy chain portion or light chain portion) can be created by introducing one or more nucleotide substitutions, additions or deletions into the nucleotide sequence of the immunoglobulin such that one or more amino acid substitutions, additions or deletions are introduced into the encoded protein. Mutations may be introduced by standard techniques, such as site-directed mutagenesis and PCR-mediated mutagenesis. Preferably, conservative amino acid substitutions are made at one or more non-essential amino acid residues.

### Manufacturing Methods

Polynucleotides encoding the anti-TSLP Fabs or antigen binding fragments are typically inserted in an expression vector for introduction into host cells that may be used to produce the desired quantity of the Fab. As such, an expression vector comprising polynucleotides encoding a Fab defined herein, and a host cell comprising said expression vector are encompassed herein.

Recombinant expression of a Fab or antigen binding fragment requires construction of an expression vector containing a polynucleotide that encodes the Fab. Once a polynucleotide encoding the Fab of the disclosure has been obtained, the vector for the production of the Fab may be produced by recombinant DNA technology using techniques well known in the art.

DNA sequences that encode the Fab may be made, either simultaneously or separately, using reverse transcriptase and DNA polymerase in accordance with well-known methods. PCR may be initiated by consensus constant region primers or by more specific primers based on the published DNA and amino acid sequences. PCR also may be used to isolate DNA clones encoding antibody variable light and heavy chains. In this case the libraries may be screened by consensus primers or larger homologous probes, such as mouse constant region probes.

Thus, methods for preparing a protein by expressing a polynucleotide containing a Fab encoding nucleotide sequence are described herein. Methods that are well known to those skilled in the art can be used to construct expression vectors containing anti-TSLP Fab coding sequences and appropriate transcriptional and translational control signals. These methods include, for example, in vitro recombinant DNA techniques, synthetic techniques, and in vivo genetic recombination. The disclosure, thus, provides replicable vectors comprising a nucleotide sequence encoding a Fab of the disclosure operably linked to a promoter.

For the purposes of this disclosure, numerous expression vector systems may be employed. For example, one class of vector utilizes DNA elements that are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells that have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. Additional elements may also be needed for optimal synthesis of mRNA. These elements may include signal sequences, splice signals, as well as transcriptional promoters, enhancers, and termination signals.

Any expression vector that is capable of eliciting expression in eukaryotic cells may be used in the present disclosure. Examples of suitable vectors include, but are not limited to plasmids pcDNA3, pHCMV/Zeo, pCR3.1, pEF 1/His, pIND/GS, pRc/HCMV2, pSV40/Zeo2, pTRACER-HCMV, pUB6/V5-His, pVAXI, and pZeoSV2 (available from Invitrogen, San Diego, Calif.), and plasmid pCI (available from Promega, Madison, Wis.). In general, screening large numbers of transformed cells for those that express

More generally, once the vector or DNA sequence encoding the Fab has been prepared, the expression vector may be introduced into an appropriate host cell. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway (1988) “Mammalian Expression Vectors” in Vectors, ed. Rodriguez and Denhardt (Butterworths, Boston, Mass.), Chapter 24.2, pp. 470-472. Typically, plasmid introduction into the host is via electroporation. The host cells harboring the expression construct are grown under conditions appropriate to the production of the anti-TSLP Fab, and assayed for protein synthesis. Exemplary assay techniques include enzyme-linked immunosorbent assay (ELISA), radioimmunoassay (RIA), or fluorescence-activated cell sorter analysis (FACS), immunohistochemistry and the like.

The expression vector is transferred to a host cell by conventional techniques, and the transfected cells are then cultured by conventional techniques to produce an anti-TSLP Fab for use in the methods described herein. Thus, the disclosure includes host cells containing a polynucleotide encoding a Fab of the disclosure, for example a heavy or light chain, or a variable heavy or variable light chain, operably linked to a heterologous promoter.

In one instance, there is provided a culture medium comprising said host cell. In one instance, there is provided a fermentation vessel comprising said culture medium.

In some instances the culture medium and the fermentation vessel are suitable for carrying out the method of producing a Fab defined herein.

A variety of host-expression vector systems may be utilized to express the anti-TSLP Fabs described herein. Such host-expression systems represent vehicles by which the coding sequences of interest may be produced and subsequently purified, but also represent cells that may, when transformed or transfected with the appropriate nucleotide coding sequences, express a molecule of the disclosure in situ. These include, but are not limited to, microorganisms such as bacteria (e.g., E. coli, B. subtilis) transformed with recombinant bacteriophage DNA, plasmid DNA or cosmid DNA expression vectors containing coding sequences; yeast (e.g., Saccharomyces, Pichia) transformed with recombinant yeast expression vectors containing coding sequences; insect cell systems infected with recombinant virus expression vectors (e.g., baculovirus) containing coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing coding sequences; or mammalian cell systems (e.g., COS, CHO, BLK, 293, 3T3 cells) harbouring recombinant expression constructs containing promoters derived from the genome of mammalian cells (e.g., metallothionein promoter) or from mammalian viruses (e.g., the adenovirus late promoter; the vaccinia virus 7.5K promoter).

Bacterial cells such as Escherichia coli, and more preferably, eukaryotic cells are used for the expression of a Fabs. For example, mammalian cells such as Chinese hamster ovary cells (CHO), in conjunction with a vector such as the major intermediate early gene promoter element from human cytomegalovirus is an effective expression system for antibodies (Foecking et al., Gene 45: 101 (1986); Cockett et al, Bio/Technology 8:2 (1990)).

The host cell line used for protein expression is often of mammalian origin; those skilled in the art are credited with ability to preferentially determine particular host cell lines that are best suited for the desired gene product to be expressed therein. Exemplary host cell lines include, but are not limited to, CHO (Chinese Hamster Ovary), DG44 and DUXB13 (Chinese Hamster Ovary lines, DHFR minus), HELA (human cervical carcinoma), CVI (monkey kidney line), COS (a derivative of CVI with SV40 T antigen), VERO, BHK (baby hamster kidney), MDCK, 293, WI38, R1610 (Chinese hamster fibroblast) BALBC/3T3 (mouse fibroblast), HAK (hamster kidney line), SP2/0 (mouse myeloma), P3. times.63-Ag3.653 (mouse myeloma), BFA-lclBPT (bovine endothelial cells), RAJI (human lymphocyte) and 293 (human kidney). Host cell lines are typically available from commercial services, the American Tissue Culture Collection or from published literature.

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used.

For long-term, high-yield production of recombinant proteins, stable expression is preferred. For example, cell lines that stably express the anti-TSLP Fab may be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which stably express the anti-TSLP Fab.

A number of selection systems may be used, including, but not limited to, the herpes simplex virus thymidine kinase (Wigler et al., Cell 13:223 (1977)), hypoxanthine-guanine phosphoribosyltransferase (Szybalska and Szybalski, Proc. Natl. Acad. Sci. USA 48:202 (1992)), and adenine phosphoribosyltransferase (Lowy et al., Cell 22:817 (1980)) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78: 1521 (1981)); gpt, which confers resistance to mycophenolic acid (Mulligan and Berg, Proc. Natl. Acad. Sci. USA 78:2012 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 52:573-596 (1993); Mulligan, Science 260:926-932 (1993); and Morgan and Anderson, Ann. Rev. Biochem. 62: 191-217 (1993); TIB TECH 13(5): 155-215 (May, 1993); and hygro, which confers resistance to hygromycin (Santerre et al., Gene 30: 141 (1984). Methods commonly known in the art of recombinant DNA technology which can be used are described in Ausubel et al. (1993) Current Protocols in Molecular Biology (John Wiley & Sons, NY); Kriegler (1990) “Gene Transfer and Expression” in A Laboratory Manual (Stockton Press, NY); Dracopoli et al. (eds) (1994) Current Protocols in Human Genetics (John Wiley & Sons, NY) Chapters 12 and 13; Colberre-Garapin et al. (1981) J. Mol. Biol. 150: 1, which are incorporated by reference herein in their entireties.

The expression levels of a Fab can be increased by vector amplification (for a review, see Bebbington and Hentschel (1987) “The Use of Vectors Based on Gene Amplification for the Expression of Cloned Genes in Mammalian Cells in DNA Cloning” (Academic Press, NY) Vol. 3. When a marker in the vector system expressing the anti-TSLP Fab is amplifiable, increase in the level of inhibitor present in culture of host cell will increase the number of copies of the marker gene. Since the amplified region is associated with the anti-TSLP Fab gene, production of the anti-TSLP Fab will also increase (Crouse et ah, Mol. Cell. Biol. 3:251 (1983)).

In vitro production allows scale-up to give large amounts of the desired polypeptides. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or (immuno-)affinity chromatography, e.g., after preferential biosynthesis of a synthetic hinge region polypeptide or prior to or subsequent to the HIC chromatography step described herein.

Genes encoding anti-TSLP Fabs of the disclosure can also be expressed in non-mammalian cells such as insect, bacteria or yeast or plant cells. Bacteria that readily take up nucleic acids include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the heterologous polypeptides typically become part of inclusion bodies. The heterologous polypeptides must be isolated, purified and then assembled into functional molecules. Where tetravalent forms of antibodies are desired, the subunits will then self-assemble into tetravalent antibodies (WO 02/096948 A2).

In bacterial systems, a number of expression vectors may be advantageously selected depending upon the use intended for the anti-TSLP Fab being expressed. For example, when a large quantity of such a protein is to be produced, for the generation of pharmaceutical compositions of a Fab, vectors which direct the expression of high levels of fusion protein products that are readily purified may be desirable. Such vectors include, but are not limited, to the E. coli expression vector pUR278 (Ruther et al., EMBO J. 2: 1791 (1983)), in which the coding sequence may be ligated individually into the vector in frame with the lacZ coding region so that a fusion protein is produced; pIN vectors (Inouye and Inouye, Nucleic Acids Res. iJ:3101-3109 (1985); Van Heeke and Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In addition to prokaryotes, eukaryotic microbes may also be used. Saccharomyces cerevisiae, or common baker's yeast, is the most commonly used among eukaryotic microorganisms although a number of other strains are commonly available, e.g., Pichia pas tons.

For expression in Saccharomyces, the plasmid YRp7, for example, (Stinchcomb et ah, Nature 282:39 (1979); Kingsman et al, Gene 7: 141 (1979); Tschemper et al, Gene 10: 151 (1980)) is commonly used. This plasmid already contains the TRP1 gene, which provides a selection marker for a mutant strain of yeast lacking the ability to grow in tryptophan, for example ATCC No. 44076 or PEP4-1 (Jones, Genetics 85: 12 (1977)). The presence of the trpl lesion as a characteristic of the yeast host cell genome then provides an effective environment for detecting transformation by growth in the absence of tryptophan.

In an insect system, Autographa californica nuclear polyhedrosis virus (AcNPV) is typically used as a vector to express foreign genes. The virus grows in Spodoptera frugiperda cells. The Fab coding sequence may be cloned individually into non-essential regions (for example the polyhedrin gene) of the virus and placed under control of an AcNPV promoter (for example the polyhedrin promoter).

Once a Fab of the disclosure has been recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Alternatively, a preferred method for increasing the affinity of antibodies of the disclosure is disclosed in U.S. Patent Application Publication No. 2002 0123057 A1.

### Methods of Treatment

The disclosure also provides methods of treatment comprising administering to a subject in need thereof a therapeutically effective amount of an anti-TSLP Fab or a pharmaceutical composition described herein. In some instances the method is for the treatment of a TSLP-related condition.

The disclosure also provides the anti-TSLP Fabs or pharmaceutical compositions described herein for use in therapy. In some instances, the therapy is the treatment of a TSLP-related condition.

The disclosure also provides the use of the anti-TSLP Fabs or pharmaceutical compositions in the manufacture of a medicament for use in the treatment of a disease. In some instances, the disease is a TSLP-related condition.

The disclosure also provides for the use of the anti-TSLP Fabs or pharmaceutical compositions in therapy. In some instances, the therapy is the treatment of a TSLP-related condition.

In some instances, the TSLP-related condition is a TSLP-related inflammatory condition. In some instances, the TSLP-related inflammatory condition is selected from asthma, sepsis, septic shock, atopic dermatitis, allergic rhinitis, allergic rhinosinusitis, allergic conjunctivitis, eosinophilic esophagitis, rheumatoid arthritis, chronic obstructive pulmonary disease (COPD), asthma, COPD overlap syndrome (ACOS), chronic bronchitis, emphysema, chronic rhinosinusitis with or without nasal polyps, vasculitis, GvHD, uveitis, chronic idiopathic urticaria, sinusitis or pancreatitis.

In some instances, the TSLP-related inflammatory condition is asthma.

Asthma is a complex and heterogenous inflammatory disease of the airways characterized by variable and recurring symptoms, reversible airflow obstruction, and bronchospasm.

Asthma symptoms can include wheezing, coughing, chest tightness, and shortness of breath. Symptoms can be triggered by exposure to allergens or irritants. Asthma may be classified as atopic (extrinsic) or non-atopic (intrinsic), based on whether symptoms are precipitated by allergens (atopic) or not (non-atopic). An acute asthma exacerbation is commonly referred to as an “asthma attack”. Further signs which can occur during an asthma attack include the use of accessory muscles of respiration (sternocleidomastoideoalene muscles of the neck), there may be a paradoxical pulse (a pulse that is weaker during inhalation and stronger during exhalation), and over-inflation of the chest. A blue color of the skin and nails may occur from lack of oxygen. In addition to these positive therapeutic responses, The subject undergoing treatment with an anti-TSLP Fab may experience a beneficial effect or an improvement in one or more of these symptoms associated with the disease.

Clinical response can be assessed using screening techniques such as magnetic resonance imaging (MRI) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, chromatography, and the like.

The Fab disclosed herein can be used in combination with any known therapy for inflammatory diseases, including any agent or combination of agents that are known to be useful, or which have been used or are currently in use, for treatment of inflammatory diseases, e.g., asthma or COPD. Exemplary active agents that can be administered in combination with a Fab described herein include, but are not limited to, inhaled corticosteroids (ICS), bronchodilators (including long-acting beta agonists (LABA), long-acting anti-muscarinic agonists (LAMA), short-acting beta agonist (SABA), and muscarinic ß2-agonists (MABA)), antihistamines, antileukotrienes, PDE-4 inhibitors, janus kinase inhibitors and phosphoinositide 3-kinase inhibitors.

The term “combination” refers to either a fixed combination in one dosage unit form, or a combined administration where an anti-TSLP Fab and a combination partner (e.g. another drug, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g. an anti-TSLP Fab and a combination partner, are both administered to a patient simultaneously in the form of a single entity or dosage. The term “non-fixed combination” means that the therapeutic agents, e.g., an anti-TSLP Fab and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agent.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

### Compositions

The anti-TSLP Fabs in the medical uses and methods disclosed herein may be administered to a subject in the form of a pharmaceutical composition.

In some instances, any references herein to ‘a/the anti-TSLP Fab’ may also refer to a pharmaceutical composition comprising an/the anti-TSLP Fab.

In some instances, the anti-TSLP Fab or a pharmaceutical composition thereof may be administered to a human or other animal in accordance with the aforementioned methods of treatment/medical uses in an amount sufficient to produce a therapeutic effect.

In some instances, the anti-TSLP Fab or a pharmaceutical composition thereof can be administered to such human or other animal in a conventional dosage form prepared by combining the anti-TSLP Fab with a conventional pharmaceutically acceptable carrier or diluent according to known techniques.

It will be recognized by one of skill in the art that the form and character of the pharmaceutically acceptable carrier or diluent is dictated by the amount of active ingredient with which it is to be combined, the route of administration and other well-known variables.

In some instances, pharmaceutical compositions are formulated to comprise a pharmaceutically acceptable, non-toxic, sterile carrier such as physiological saline, non-toxic buffers, preservatives and the like. In some instances the pharmaceutical composition may include sterile aqueous or non-aqueous solutions, suspensions, and emulsions. Suitable formulations for use in the therapeutic methods disclosed herein are described in Remington's Pharmaceutical Sciences (Mack Publishing Co.) 16th ed. (1980).

In some instances, the route of administration of the anti-TSLP Fab or pharmaceutical composition thereof may be, for example, oral, parenteral, by inhalation or topical. In some instances, the term parenteral as used herein includes, e.g., intravenous, intraarterial, intraperitoneal, intramuscular, subcutaneous, rectal, or vaginal administration.

In some instances, the anti-TSLP Fab or pharmaceutical composition thereof may be administered by nasal aerosol or inhalation.

In some instances, the components as recited herein for preparing said pharmaceutical composition may be packaged and sold in the form of a kit. Such a kit will In some instances have labels or package inserts indicating that the associated pharmaceutical compositions are useful for treating a subject suffering from, or predisposed to a disease or disorder.

### EXAMPLES

### Example 1

Stability studies were carried out on Tezepelumab in 1×PBS at 4° C. or 45° C. Solutions comprising 1 mg/ml Tezepelumab were stored under each condition for two weeks.

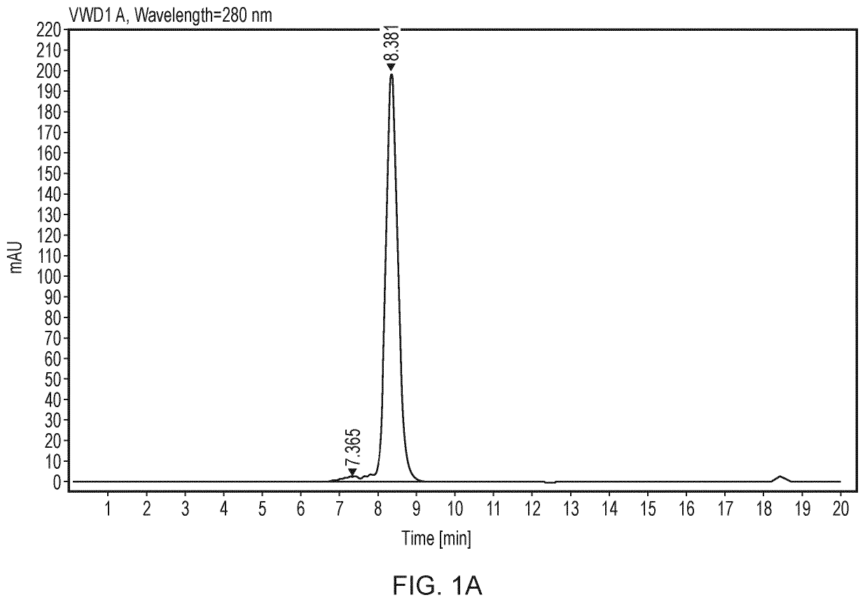

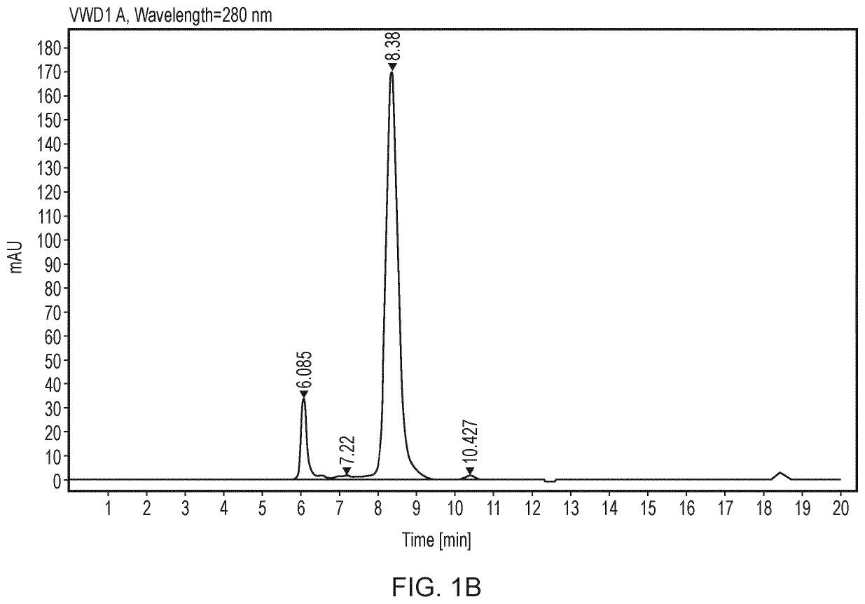

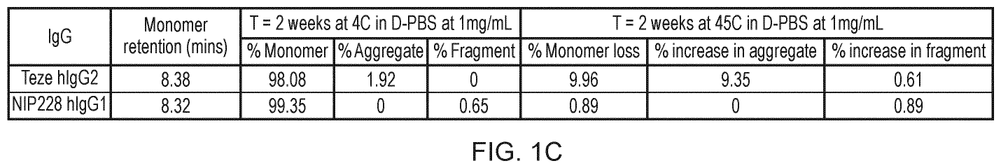

The results show that 10% loss of monomer was observed after 2 weeks at 45° C. (FIG. 1) in D-PBS. % monomer loss, % aggregation and % fragmentation were calculated by quantifying area under the curve (AUC) from HP-SEC chromatograms. Peaks were assigned as monomer, aggregate or fragmentation product based on elution volume. The AUC can be calculated using standard analytical tools provided with HP SEC analysis software.

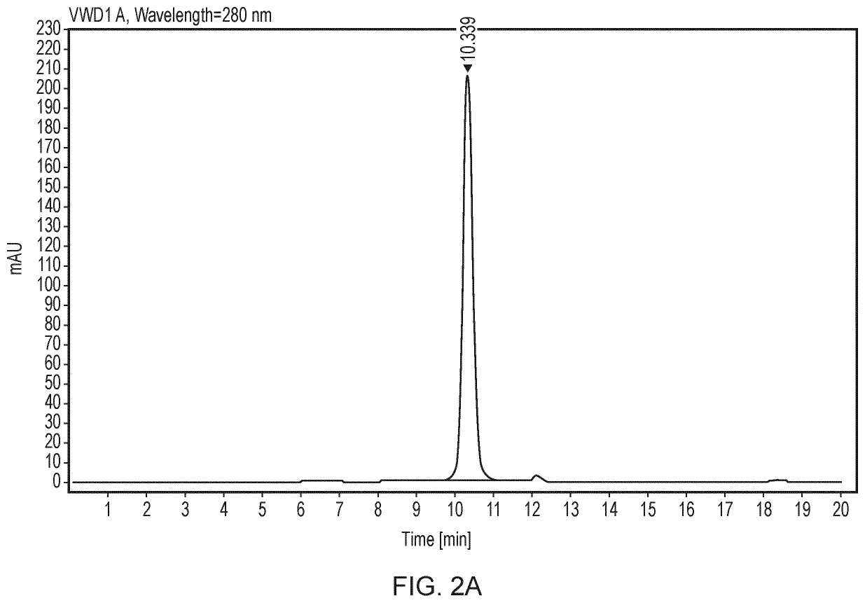

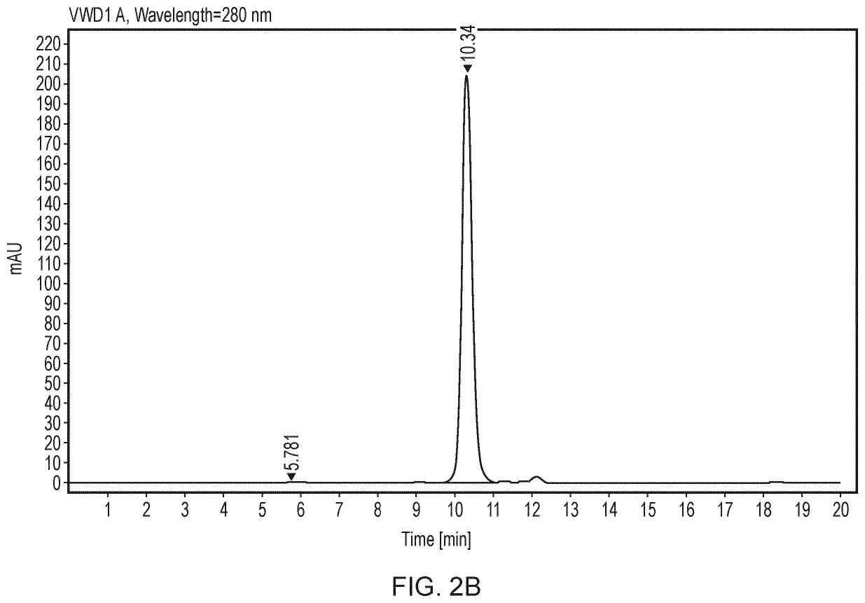

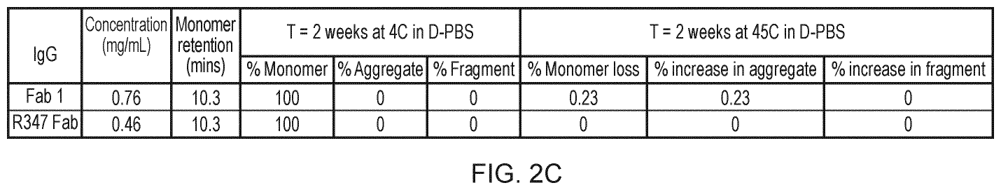

A Fab was constructed comprising Tezepelumab heavy and light complementarity determining regions (CDRs) with multiple mutations in the CH1 domain. This Fab is referred to herein as Fab 1. Fab 1 stability was analysed as described above for Tezepelumab. The results are shown in FIG. 2.

Surprisingly, Fab 1 showed improved stability properties under accelerated stability study conditions (1×PBS at 45° C.) when stored at 0.8 mg/ml for two weeks.

### Example 2—Fab1 Binds to Hu and Cyno TSLP with pM Affinity

### Affinity of Fab₁ Binding to TSLP Determined by BIAcore

The specificity and affinity of Fab 1 for recombinant mammalian cell-expressed human and cyno TSLP were determined using a Biacore 8K SPR instrument (GE Healthcare, Little Chalfont, Bucks, UK).

S Series C1 biosensor chips, amine coupling kits, hepes buffered saline-based buffers and regeneration buffers were obtained from GE Healthcare and used according to the manufacturer's instructions. Streptavidin surfaces were prepared using lyophilized streptavidin that was reconstituted with D-PBS. Briefly, streptavidin was diluted to 4 μg mL-1 in 10 mM sodium acetate pH 4.5 and covalently immobilized to three flow cell surfaces of a S Series C1 biosensor chip by standard amine coupling methods. A final streptavidin surface of 170 response units (RUs) was achieved. The amine coupling reagents were also used to prepare a control blank surface, with no immobilized streptavidin, to serve as a reference surface within each flow cell. N-terminally tagged biotinylated TSLP (human and cyno) were then titrated onto each streptavidin surface to enable <100 RUs of Fab1 binding at saturation (Rmax). The low level of analyte binding ensured that mass-transport induced artefacts were minimized, especially when combined with the relatively fast, 50 μL min-1 assay flow-rates used during the kinetics measurement steps. Dilutions (Multi-Cycle Kinetics) of monomerized Fab1 (2-fold dilutions in HBS-EP+ buffer ranging between 1.25 and 20 nM) were injected, at a 50 μL min-1 assay flowrate, for 2 minutes of association and 10 minutes dissociation. Multiple buffer-only injections were made under the same conditions throughout the experiment to allow for double reference processing of the final sensorgram sets.

The chip surface was fully regenerated by flowing two 30 second pulses of 10 mM glycine pH 1.7. Binding affinity and kinetics were determined using 1:1 Langmuir model.

The results shown in Table 1 demonstrate that Fab₁ binds to immobilized hu and cyno TSLP with similar affinities (within 2-fold; 46 pM and 88 pM, respectively).

TABLE 1
Affinity of Fab1 for Human and Cynomolgus TSLP using BIAcore

| Analyte | kₐ (M⁻¹s⁻¹) | k_{d} (s⁻¹) | K_{D} (pM) |
|---|---|---|---|
| Human TSLP | 2.39E6 | 1.11E−4 | 46.3 |
| cyno TSLP | 1.75E6 | 1.55E−4 | 88.4 |

Binding Affinity determined by Kinetic Exclusion Assay (KinExA).

The solution phase binding affinity (K_{D}) of Fab 1 for human and cyno TSLP was also determined using a KinExA 3200 instrument (Sapidyne Instruments, Boise, Idaho, USA) and the resulting data was processed using the KinExA Pro software version 4.1.11. The KinExA methodology has been reviewed (Darling and Brault, 2004).

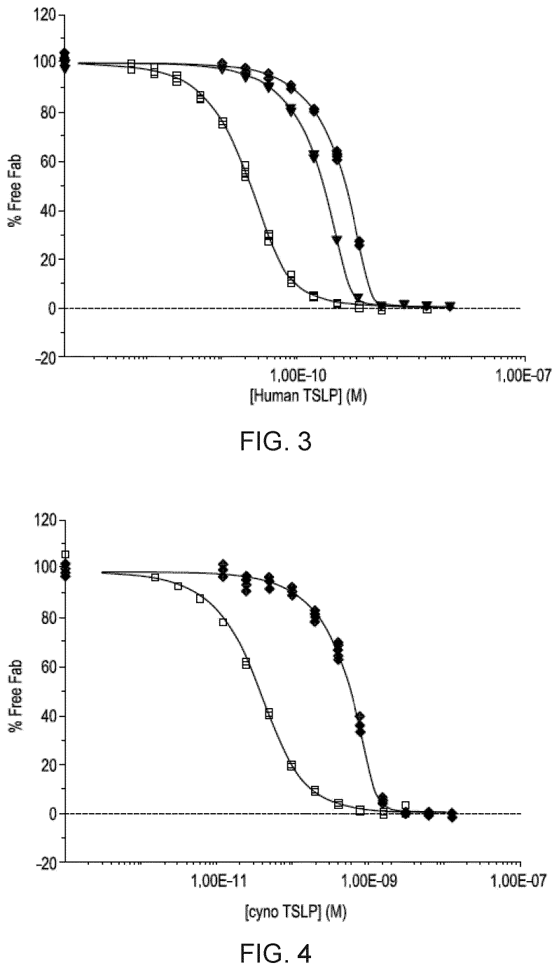

Fab 1 was pre-mixed with varying concentrations of each of hu and cyno TSLP until equilibrium was reached (at least 12 concentrations of each hu and cyno TSLP were prepared using a 2-fold serial dilution method). The amount of free Fab 1 was then measured using the KinExA instrument by capturing free Fab using hu TSLP-coated beads, washing away unbound material and detecting bound Fab 1 fluorometrically using a commercial, species-specific antibody (Alexa Fluor 647 labelled mouse anti-Human Heavy and Light chain specific antibody (Jackson Immunoresearch 209-605-088)). The K_{D} of Fab 1 for hu TSLP was extracted by global 1:1 fit to three datasets, derived from hu TSLP titrations into 1000 pM (filled diamonds), 500 pM (inverted filled triangles) or 40 pM (open squares) fixed Fab₁ concentration solutions (FIG. 3). The K_{D} of Fab 1 for cyno TSLP was extracted by global 1:1 fit to two datasets, derived from cyno TSLP titrations into 1000 pM (filled diamonds) or 40 pM (open squares) fixed Fab₁ concentration solutions (FIG. 4).

The amount of free Fab 1 detected at each hu and cyno TSLP concentration was plotted against the titrated concentration of TSLP (FIGS. 3 and 4, respectively). The KinExA software was used to calculate the equilibrium dissociation constant (KD). The results shown in Table 2 demonstrate that Fab₁ binds to human TSLP with a 1.7-fold higher affinity than it binds to cyno TSLP in free solution.

TABLE 2
Soluble Phase Affinity of Fab₁ for
hu and cyno TSLP using KinExA

| Ligand | Affinity (K_{D}) pM |
|---|---|
| Human TSLP | 8.0 (95% Conf. Int. 6.27-10.01 pM) |
| cyno TSLP | 13.6 (95% Conf. Int. 9.07-19.22 pM) |

### Example 3—Fab1 and Tezepelumab Bind to TSLP with Similar Binding Characteristics

The binding characteristics of Fab 1 to hu TSLP were directly compared with tezepelumab.

The in vitro binding potency of Fab 1 was determined using a homogeneous fluorescence resonance energy transfer (FRET) Homogeneous Time-Resolved Fluorescence (HTRF®, Cisbio International) based TSLP: mAb-binding assay. Streptavidin cryptate was used for the detection of biotinylated TSLP. In brief, samples of unlabeled Fab 1 were titrated into the HTRF assay to compete with DyLight-labelled tezepelumab for binding to biotinylated His-Avi hu TSLP. A competition assay was also preformed using unlabeled tezepelumab and DyLight-labelled tezepelumab as a positive control.

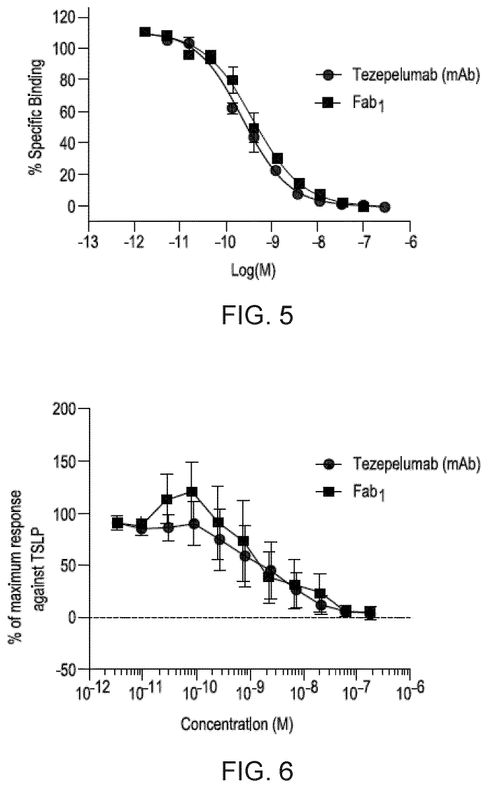

The results show that Fab 1 competes for binding to huTSLP with tezepelumab and binds to hu TSLP with a similar potency as tezepalumab (IC50: Fab1—0.38 nM; tezepelumab—0.23 nM—FIG. 5).

### Example 4—Fab 1 Neutralizes TSLP Activity in a Peripheral Blood Mononuclear Cell (PBMC) Assay

It was next determined whether Fab 1 binding to TSLP has functional blocking activity in a primary cell assay by measuring TSLP-induced CCL17 release from PBMCs upon treatment with Fab 1.

Blood was obtained from healthy donors under the blood donor program established at MedImmune, Cambridge, UK. Peripheral blood mononuclear cells were isolated by a standard procedure using a ficoll gradient. Briefly, 20 mls of blood diluted with PBS (10 ml blood:30 ml PBS) were layered onto 15 ml ficoll. Tubes were spun at 400 g for 40 mins at room temperature without brake. PBMC layers were collected and cells washed twice with 50 ml PBS. PBMCs were counted using a haemocytometer and trypan blue to exclude dead cells and resuspended in culture media (RPMI with 10% fetal calf serum and 1% penicillin/streptomycin) before plating into a 96-well plate. Cells were stimulated with TSLP (0.5 ng/ml) in the presence of the TSLP-binding antibody fragment Fab1, for 48 h. Assays were also performed using the TSLP-binding antibody tezepelumab, as a positive control. After 48 h, supernatants were removed and assayed for CCL17 production using an R&D duoset ELISA, according to the manufacturer's protocol. Experiments were performed using six donors in three independent experiments.

The results show that Fab 1 inhibited CCL17 production from PBMCs with an IC₅₀ of 1.39 nM (FIG. 6).

Sequences

SEQ ID NO: 1 (Fab 1 Heavy Chain)
QMQLVESGGGVVQPGRSLRLSCAASGFTFRTYGMHWVRQAPGKGLEWVAV
IWYDGSNKHYADSVKGRFTITRDNSKNTLNLQMNSLRAEDTAVYYCARAP
QWELVHEAFDIWGQGTMVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKRVEPKSCDK
SEQ ID NO: 2 (Fab 1 Light Chain)
SYVLTQPPSVSVAPGQTARITCGGNNLGSKSVHWYQQKPGQAPVLVVYDD
SDRPSWIPERFSGSNSGNTATLTISRGEAGDEADYYCQVWDSSSDHVVFG
GGTKLTVLGQPKAAPSVTLFPPSSEELQANKATLVCLISDFYPGAVTVAW
KADSSPVKAGVETTTPSKQSNNKYAASSYLSLTPEQWKSHRSYSCQVTHE
GSTVEKTVAPTECS

1. A kit comprising a Fab wherein the Fab comprises a heavy chain comprising the amino acid sequence set forth in SEQ ID NO:1 and a light chain comprising the amino acid sequence set forth in SEQ ID NO:2.

2. A kit comprising a Fab wherein the Fab comprises a heavy chain having the amino acid sequence set forth in SEQ ID NO:1 and a light chain having the amino acid sequence set forth in SEQ ID NO:2.

3. A kit comprising a Fab wherein the Fab comprises a heavy chain consisting of the amino acid sequence set forth in SEQ ID NO:1 and a light chain consisting of the amino acid sequence set forth in SEQ ID NO:2.

4. The kit according to claim 1, wherein the Fab is an IgGI Fab.

5. The kit according to claim 1, wherein the Fab is stable at 45° C. for a period of at least two weeks.

6. The kit according to claim 5, wherein the Fab is stable in 1×PBS at a concentration of 0.8 mg/ml.

7. The kit according to claim 1, wherein the Fab shows less than 10% monomer loss following incubation at 45° C. in 1×PBS for 2 weeks, wherein monomer is determined by HP-SEC.

8. A kit comprising a pharmaceutical composition wherein the pharmaceutical composition comprises the Fab of claim 1.

9. A nucleic acid encoding the Fab of claim 1.

10. A vector comprising the nucleic acid of claim 9.

11. A host cell comprising the nucleic acid of claim 9.

12. A method of producing the Fab of claim 1 comprising culturing the host cell, expressing the Fab and purifying the Fab.

13. A Fab obtainable from the method of claim 12.

14. A kit comprising a Fab according to claim 1 for therapy.

15. A kit comprising a Fab according to claim 1, for a method of treating a TSLP-related condition.

16. The kit comprising a Fab according to claim 15, wherein the TSLP-related condition is asthma.

17. A method of treating a subject in need thereof, the method comprising administering to the subject a therapeutically effective amount of a Fab comprising, having, or consisting of a heavy chain comprising the amino acid sequence set forth in SEQ ID NO:1 and a light chain comprising, having, or consisting of the amino acid sequence set forth in SEQ ID NO:2, wherein the subject has asthma, sepsis, septic shock, rheumatoid arthritis, COPD overlap syndrome (ACOS), chronic bronchitis, emphysema, chronic rhinosinusitis with or without nasal polyps, vasculitis, GvHD, uveitis, chronic idiopathic urticaria, sinusitis or pancreatitis.

18. The method according to claim 17, wherein the Fab is an IgG1 Fab.


SEQUENCE LISTING (2 sequences)

SEQ ID NO: 1 - protein, 227 aa, Artificial Sequence
  FAB1 HEAVY CHAIN
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 2 - protein, 214 aa, Artificial Sequence
  FAB1 LIGHT CHAIN
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS